(19) United States
(12) Patent Application Publication
(10) Pub. No.: US 2025/0117685 A1
(43) Pub. Date: Apr. 10, 2025

(54) ITERATIVE MACHINE LEARNING INTERATOMIC POTENTIAL (MLIP) TRAINING METHODS

(71) Applicant: Robert Bosch GmbH, Stuttgart (DE)

(72) Inventors: Shao-Chun LEE, Champaign, IL (US); Mordechai KORNBLUTH, Brighton, MA (US); Nicola MOLINARI, Cambridge, MA (US); Daniil KITCHAEV, Brookline, MA (US)

(21) Appl. No.: 18/377,520

(22) Filed: Oct. 6, 2023

(51) Int. Cl. G06N 20/00

(52) U.S. Cl. CPC G06N 20/00

(57) ABSTRACT

An iterative machine learning interatomic potential (MLIP) training method. The training method includes training a first multiplicity of first MLIP models in a first iteration of a training loop. The training method further includes training a second multiplicity of second MLIP models in a second iteration of the training loop in parallel with the first training step. The training method also includes combining the first MLIP models and the second MLIP models to create an iteratively trained MLIP configured to predict one or more values of a material. The MLIP may be a Gaussian Process (GP) based MLIP (e.g., FLARE). The MLIP may be a graph neural network (GNN) based MLIP (e.g., NequIP or Allegro).

### TECHNICAL FIELD

This invention relates generally to iterative machine learning interatomic potential (MLIP) training methods. The MLIP may be a Gaussian Process (GP) based MLIP (e.g., FLARE) and/or a graph neural network (GNN) based MLIP (e.g., NequIP or Allegro).

### BACKGROUND

Ab-initio calculations (e.g., density functional theory, otherwise referred to as DFT) have been used to obtain accurate estimations of interaction energies of a material at an atomistic scale. These ab-initio calculations are computationally expansive, thereby having a high magnitude of computational cost. Machine learned interatomic potentials (MLIPs) may be used to develop shorter-range models that can infer interaction energies with accuracy close to ab-initio calculations while reducing the computational costs by several orders of magnitude. MLIPs have shown promise as a compromise between accuracy and computational efficiency.

### SUMMARY

According to one embodiment, an iterative machine learning interatomic potential (MLIP) training method is disclosed. The training method includes training a first multiplicity of first MLIP models in a first iteration of a training loop. The training method further includes training a second multiplicity of second MLIP models in a second iteration of the training loop in parallel with the first training step. The training method also includes combining the first MLIP models and the second MLIP models to create an iteratively trained MLIP configured to predict one or more values of a material (e.g., atomic level and/or structural level values). A non-limiting example of a structural level value is total energy. The MLIP may be a Gaussian Process (GP) based MLIP (e.g., FLARE). The MLIP may be a graph neural network (GNN) based MLIP (e.g., NequIP or Allegro) or other deep learning based MLIP.

According to another embodiment, an iterative machine learning interatomic potential (MLIP) training method is disclosed. The training method includes receiving one or more structures associated with a material. The training method further includes training first MLIP models in a first training block using a first multiplicity of systems and the one or more structures associated with the material. The training method also includes training second MLIP models in a second training block using a second multiplicity of systems. The training method also includes outputting one or more datasets from the second training block. The one or more datasets may be configured to predict one or more values of a material (e.g., atomic level and/or structural level values). A non-limiting example of a structural level value is total energy.

According to yet another embodiment, an iterative machine learning interatomic potential (MLIP) training method is disclosed. The training method includes receiving one or more datasets associated with a material. The method further includes training first MLIP models in a first training block using a multiplicity of first sessions and the one or more datasets associated with the material. The method also includes training second MLIP models in a second training block using a second session. The method also includes outputting one or more structures from the second training block. The one or more structures may be configured to predict one or more values of a material (e.g., atomic level and/or structural level values). A non-limiting example of a structural level value is total energy.

### BRIEF DESCRIPTION OF THE DRAWINGS

FIG. 1 depicts a workflow of an iterative machine learning interatomic potential (MLIP) training method according to one or more embodiments.

FIGS. 2A, 2B, and 2C depict models representing an example of active learning with an enhanced sampling strategy between two fluorocarbon chains.

FIG. 3 is a schematic diagram of the functions of a block-level directory of an iterative GP-based MLIP training method according to one or more embodiments.

FIG. 4 depicts a workflow of an iterative MLIP training method according to one or more embodiments.

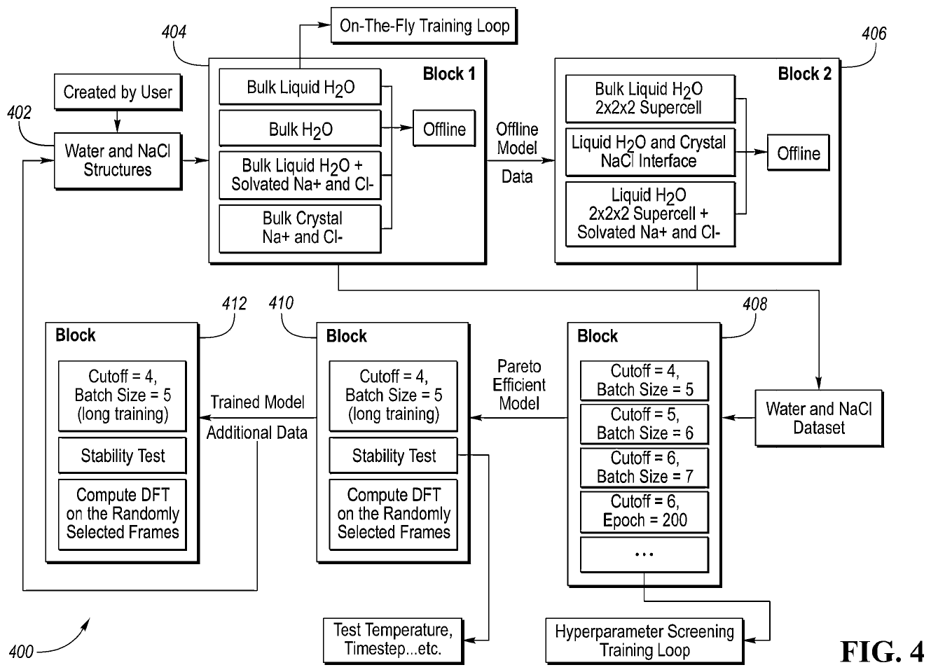

FIG. 5 is a schematic diagram of the functions of a block-level directory of an iterative GNN-based MLIP training method according to one or more embodiments.

FIG. 6 is a schematic diagram of a computing platform that may be utilized to implement the iterative MLIP training methods of one or more embodiments.

### DETAILED DESCRIPTION

As required, detailed embodiments of the present invention are disclosed herein; however, it is to be understood that the disclosed embodiments are merely exemplary of the invention that may be embodied in various and alternative forms. The figures are not necessarily to scale; some features may be exaggerated or minimized to show details of particular components. Therefore, specific structural and functional details disclosed herein are not to be interpreted as limiting, but merely as a representative basis for teaching one skilled in the art to variously employ the present invention.

The term “substantially” may be used herein to describe disclosed and/or claimed embodiments. The term “substantially” may modify a value or relative characteristic disclosed or claimed in the present disclosure. In such instances, “substantially” may signify that the value or relative characteristic it modifies is within±0%, 0.1%, 0.5%, 1%, 2%, 3%, 4%, 5% or 10% of the value or relative characteristic.

The validity of machine learning interatomic potentials (MLIPs) relies on interpolation of structures available in the training data used by the MLIPs, while the configurational space of the training data depends on the method used to generate the training data. Ab-initio molecular dynamics (AIMD) may be used to generate training data for an MLIP. However, the AIMD-based procedure is highly inefficient because it often contains highly redundant data and poor coverage of available system configurations.

In recent years, several MLIPs have emerged to improve accuracy and data efficiency. For instance, FLARE and Allegro have been developed by the Materials Intelligence Group (MIR) at Harvard University. Other groups have also developed MLIPs based on deep learning, graph neural networks, graph transformers, and other architectures. As another example, NequIP is an open-source code for building E(3)-equivariant interatomic potentials. These MLIPs may train on a relatively large number of training structures at high-fidelity computation levels to predict interatomic forces or environments. For instance, the MLIP training may include the following steps: (a) generating a set of structures; (b) using DFT or other high-fidelity approach to compute atomic values (e.g., total energy, atomic forces, stresses, charge distribution, and/or polarization) and/or structural level values; and (c) train the MLIP to correctly predict one or more atomic values and/or structural level values. For example, the total energy may be predicted and the atomic forces and/or stresses may be computed by taking a derivative with respect to the atomic positions and/or strains, respectively.

The FLARE MLIP drives molecular dynamics (MD) by a Gaussian Process (GP) based MLIP and generates training data only when the estimated uncertainty exceeds a given criteria. In the GP formalism, it is possible to identify an uncertainty of a given prediction, based on the difference between the atomic environment in the training data versus the atomic environment at the time of the inference. In this way, the FLARE MLIP learns from the data which contains new atomic configurations, hence reducing redundant ab-initio calculations on those highly overlapped configurations. This training scheme of the FLARE MLIP may be referred to as “on-the-fly” (OTF) training. On the other hand, if a pre-determined set of snapshots are fed into FLARE instead of driven by MD, the training is called “offline”. The drawbacks of FLARE are that the size of the model grows as more snapshots and/or more elements are included in the model, and the overall accuracy of the trained MLIP is lower than alternative approaches. Moreover, the memory consumption and computational efficiency may eventually degrade over time.

On the other hand, NequIP and Allegro are graph neural network (GNN) based MLIPs. NequIP and Allegro have a constant model size and inference time as a function of training data size. These types of MLIP also provide superior accuracy among known approaches. However, these methods do not have a computationally efficient and rigorous way of estimating uncertainty. As a result, a very large quantity of training data is required to achieve acceptable accuracy and stability.

An MLIP may be forced to extrapolate from its training data when it enters an atomic environment that is not represented in the training data. In this extrapolation region, a common outcome is unphysical behavior (e.g., unrealistic bond breaking, incorrect forces and energies, and/or numerical instabilities). This phenomenon may be referred to as an “instability” in the model. While in FLARE these extrapolations and instabilities may be identified and corrected using OTF training, OTF training is not an option with NequIP and Allegro, leading to a requirement that the training data is sufficiently comprehensive to avoid extrapolation in all relevant scenarios.

The mathematical characteristics of FLARE and NequIP/Allegro lead to a tradeoff between the ability to perform uncertainty-based sampling and the performance/accuracy of the final model. To avoid the weaknesses and combine the advantages of these different types of MLIPs, careful planning of the training strategy for complex atomistic systems is beneficial but challenging. What is desired is a streamlined method (e.g., workflow) that (1) automates the procedure of constructing an efficient and comprehensive training dataset; and (2) trains a stable and accurate MLIP from the dataset.

Considering the foregoing, an iterative training method (e.g., workflow) for MLIPs for atomistic-scale simulations is disclosed in one or more embodiments. The iterative training method may have one or more of the following attributes: (1) efficient training data generation for complex atomistic systems; (2) parallelized training for multiple substructures and/or hyperparameters on high-performance computing (HPC) facilities; (3) serialized training on incrementally more complex structures; and/or (4) self-consistent iterative training to identify and correct model defects. In one or more embodiments, the iterative training method streamlines procedures for building of MLIPs for complex systems and/or sampling a wide range of potential energy surfaces for different substructures. The iterative training method may facilitate the development of accurate and efficient MLIPs for computational materials and chemistry research.

The training methods of one or more embodiments may also be applied to other high-fidelity/high-speed pairups (e.g., DFT/MD pairups, quantum-chemistry/MD pairups, all-atom dynamics/coarse-grained dynamics pairups, etc.).

The iteratively trained MLIPs may be used by machine learning systems used to study physical phenomena, such as, but not limited to, ionic transport, chemical reactions, and material bulk and surface degradation in material systems, such as, devices or functional materials. Non-limiting examples of such material systems include fuel cells, surface coatings, batteries, water desalination, and water filtration.

In one or more embodiments, an iterative training method is disclosed to automate an MLIP. In certain embodiments, the iterative training method may automate a GP-based MLIP (e.g., FLARE), and may be referred to as iterative GP-based MLIP training methods. In other embodiments, the iterative training method may automate a GNN-based MLIP (e.g., NequIP and/or Allegro), and may be referred to as iterative GNN-based MLIP training methods. The iterative training methods may be implemented using one or more python packages.

In one embodiment, a method of iteratively training at least one MLIP in a loop is disclosed. Each iteration of the loop may include training a multiplicity (e.g., two or more) of different MLIP models in parallel. The method may further include combining the different MLIP models trained in parallel to create an iteratively trained MLIP. The iteratively trained MLIP may be a more accurate model than a model without training of the multiplicity of different MLIP models in parallel. The at least one MLIP may be a GP-based MLIP (e.g., FLARE). The at least one MLIP may be a GNN-based MLIP (e.g., NequIP or Allegro).

The method of iteratively training the at least one MLIP according to one or more embodiments may include an active learning component. The active learning component may include identifying a predicted confidence and/or a predicted uncertainty and calling a higher-accuracy model (e.g., DFT) if the predicted confidence falls below a predicted confidence threshold and/or the predicted uncertainty exceeds a predicted uncertainty threshold.

Two or more of the multiplicity of different MLIP models may use different hyperparameters. For example, first MLIP model(s) may use a first set of hyperparameters and second MLIP model(s) may use a second set of hyperparameters different than the first set of hyperparameters.

Two or more of the multiplicity of different MLIP models may use different starting atomic structures and/or different chemical compositions. For example, first MLIP model(s) may use a first starting atomic structure and second MLIP model(s) may use a second starting atomic structure different than the first starting atomic structure. As another example, first MLIP model(s) may use a first chemical composition and second MLIP model(s) may use a second chemical composition different than the first chemical composition. As yet another example, first MLIP model(s) may use a first starting atomic structure and a first chemical composition and second MLIP model(s) may use a second starting atomic structure different than the first starting atomic structure and a second chemical composition different than the first chemical composition.

The method of iteratively training the at least one MLIP may include a combining step where the MLIP model with the lowest error is selected. This type of selection may be patterned after a hyperparameter selection.

The method of iteratively training the at least one MLIP may include a combining step that includes retraining on a larger training set than the training set of any individual MLIP model in the multiplicity of MLIP models.

In one or more embodiments, the combining step may account for atomic environment overlap to generate a minimal-yet-descriptive set of atomistic structures and forces, allowing a more lightweight model than would be possible if all structures were used. In another embodiment, the combining step may include an automatic selection criteria based on the energy per atom in any given atomic structure to rule out unphysical configurations.

In one or more embodiments, the method of iteratively training the at least one MLIP may include enabling a later network to learn from one or more instabilities of an earlier network when there are at least two instances of the loop containing a machine learning system (e.g., a neural network) with substantially similar parameters or hyperparameters.

The multiplicity of different MLIP models trained in parallel may be prematurely terminated when the multiplicity of different MLIP models is not at least near or in a Pareto front. The Pareto front may be defined as a tradeoff between two or more parameters (e.g., speed and accuracy). A non-limiting list of parameters of interest include energy accuracy, force accuracy, stress accuracy, polarization accuracy, inference speed of the model, and GPU memory footprint of the model. The Pareto front may refer to a set of optimal solutions in a space of objective functions in multi-objective optimization problems. The set of optimal solutions may be a set of solutions that are non-dominated to each other but are superior to the rest of the solutions in the search space. The Pareto space is in principle higher than 2 dimensional (a dimension for every quantity of interest). The Pareto front may be of dimension N−1 where N is the number of quantity of interest that make up the whole space.

In one or more embodiments, a machine learning system (e.g., a neural network) in a first iteration of a loop is used to generate at least one starting structure for an active learning scheme in a second iteration of the loop.

In one or more embodiments, a first iteration of a loop may include removing datapoints based on thermodynamic relevance (e.g., structures exceeding a system-specific limit on formation energy or force magnitude).

In one or more embodiments, a method of training at least one MLIP using an active learning component of a dynamic trajectory is disclosed. The dynamic trajectory may first sample near-equilibrium structures and progress (e.g., systematically progress) to far-from-equilibrium structures.

The near-equilibrium structure may refer to a system that is substantially close to but not in a state of thermodynamic equilibrium (e.g., a state in which all macroscopic properties of a system remain constant over time without a net flow of energy within the system). The near-equilibrium structure may exhibit only substantially minor fluctuations in its properties and/or substantially insignificant changes in the system as compared to no changes in the system. The far-from-equilibrium structure may refer to a system that is substantially far from a state of thermodynamic equilibrium. The far-from-equilibrium structure may exhibit substantially large deviations from equilibrium conditions. The far-from-equilibrium structure may involve rapid and complex changes in its properties.

The dynamic trajectory may include a temperature ramping from a lower temperature advancing to through higher temperatures. The dynamic trajectory may include a biased potential to drive a collective motion of particle positions. The dynamic trajectory may include other forms of biased sampling. The bias sampling may include sampling a subset of structures from a dataset of structures of a material where the sampling is done to prioritize one or more characteristics of the structures and/or the dataset. The bias sampling may drive a particular collective motion within the material.

The iterative GP-based MLIP training method of one or more embodiments may be implemented separately from or together with the iterative GNN-based MLIP training method of one or more embodiments. An iteration of a loop disclosed herein may be referred to as a block. In one or more embodiments, a block may be serially performed. The multiplicity of trainings in parallel disclosed herein may be referred to as a system (e.g., using different chemistries and/or starting structures) or a session (e.g., using different hyperparameters).

The iterative GP-based MLIP training method of one or more embodiments may include a set of scripts configured to automate a loop of GP-based MLIP runs (e.g., FLARE runs) according to one or more of the embodiments disclosed herein.

The iterative GP-based MLIP training method may manage digital files and directories using a hierarchical order. The hierarchical order may be (1) a project level, (2) a block level, (3) a system level, and (4) an iteration level. The following discloses the functionality of each level according to one or more embodiments.

On the project level, the digital files and the digital sub-directories may be related to an application associated with a complex system that spans a broad spectrum of chemical elements and phases of materials. In one or more embodiments, a project may include a number of block-level directories. For example, solvents and/or salts may be combined into a single project.

On the block level, the digital files and the sub-directories may correspond to multiple substructures derived from an application. In one or more embodiments, the substructures may have similar length scales, a relatively simple phase and a relatively simple chemical composition. For example, a block level for solvated salts may include substructures of water, ice, ions, and ions with small solvation shells. The output of each block may include (1) a set of selected data used for training and (2) a single model that encompasses all the training data found by an active learning loop in one or more systems.

A block may start training a model from scratch (e.g., “on-the-fly from scratch”, otherwise referred to as OTF-S) when the block is not using another block's data or model. A block may start training a model with a pre-trained model output from another block (e.g., “on-the-fly from model”, otherwise referred to as OTF-M). A special type of block may include a single training from a set of known structures and forces from another block. This type of block may be referred to as “offline from scratch” (otherwise referred to as OFL-S) when the model training is started from an empty model. This type of block may be referred to as “offline from model” (otherwise referred to as OFL-M) when the training is started from a pre-trained partial model.

A block-level directory may include a multiplicity of system-level directories. The final training of the block may start with a large set of structures whose atomic environments, energies, forces, and/or stresses are known, in which the final training then selects a subset of these structures that are sufficiently distinct to provide a lightweight (e.g., relatively low-memory) model. “Distinct” may be based on the kernel of the system (e.g., atomic environment descriptors).

On the system level, the digital files and the sub-directories may correspond to a specific substructure having a fixed chemical composition (e.g., a specific instance of an atomic structure). The specific substructure may be a fixed chemical composition. In this example, a single FLARE model may be trained iteratively OTF. A system-level directory may contain a number of iteration-level directories. A special type of system-level directory may contain only a single iteration of training from the OTF training data in the same block and/or accumulated from other blocks. This system-level directory may be referred to as “offline”.

On the iteration level, the digital files and the sub-directories may be related to a single FLARE execution, which is an OTF training restarted from a previous FLARE execution. An iteration-level directory may output a single MD trajectory ready for restart and a number of selected snapshots computed by DFT.

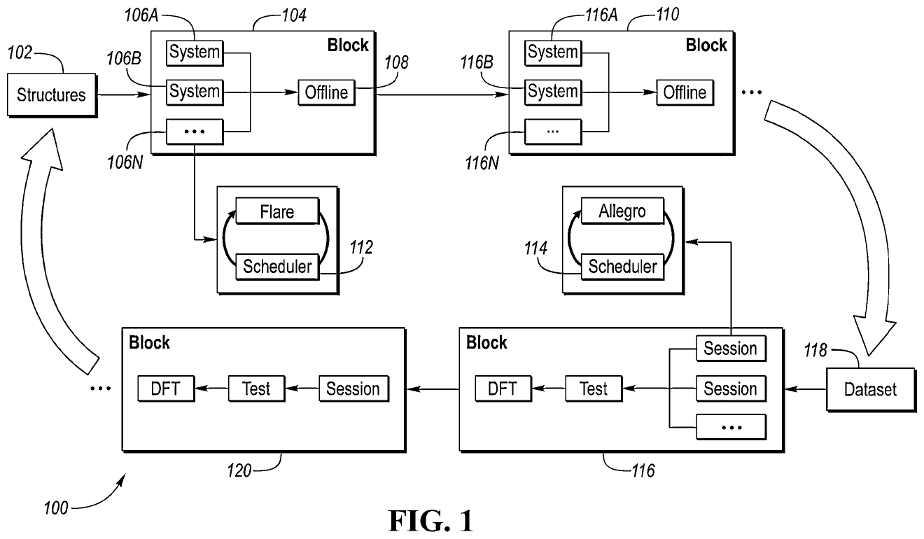

FIG. 1 depicts workflow 100 of an iterative GP-based MLIP training method (e.g., FLARE) according to one or more embodiments. Structures 102 (e.g., multiple substructures as components of a complex system) are input into block 104. One or more of the substructures may be distributed to a system-level directory as a starting point of OTF training. The system-level directory may include system 106A, 106B, and 106N. After several iterations of OTF training in each system, the data may be collected and shuffled for an offline training (e.g., 108). The offline training may eventually produce a model that learns from the data from all the substructures and saves the used and unused data to generate such model. This model can then be taken as an initial model for another block (e.g., block 110) that may have a different set of substructures. The used data from block 110 may be combined with used data from other blocks (e.g., block 104), which may generate a model that encompasses the chemical subspaces of two or more other blocks.

The iterative GP-based MLIP training method of one or more embodiments may efficiently generate training data for complex atomistic system. This training data may be realized at the system and the iteration levels. A scheduler may be created in one or more systems. As shown in FIG. 1, scheduler 112 is associated with blocks 104 and 110 and scheduler 114 is associated with block 116 and 120. The scheduler may determine a strategy for MD to drive a substructure towards an under sampled space. The strategy may be versatile and flexible, which may include ramping of the temperature of the substructure or an imposed enhanced sampling method such as collective variable dynamics. The strategy may be implemented with a computer package (e.g., PLUMED).

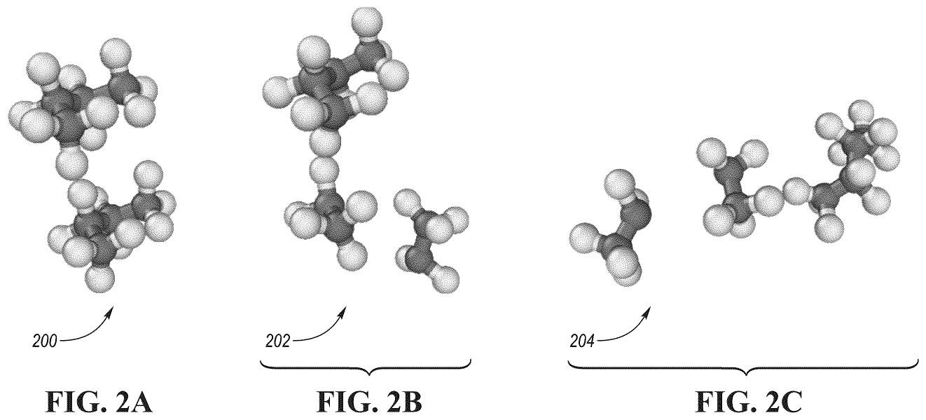

FIGS. 2A, 2B, and 2C depict models 200, 202, and 204, respectively, representing an example of active learning with an enhanced sampling strategy between two fluorocarbon chains. The iterative GP-based MLIP training method actively learns a broad spectrum of conformations and includes them in a dataset.

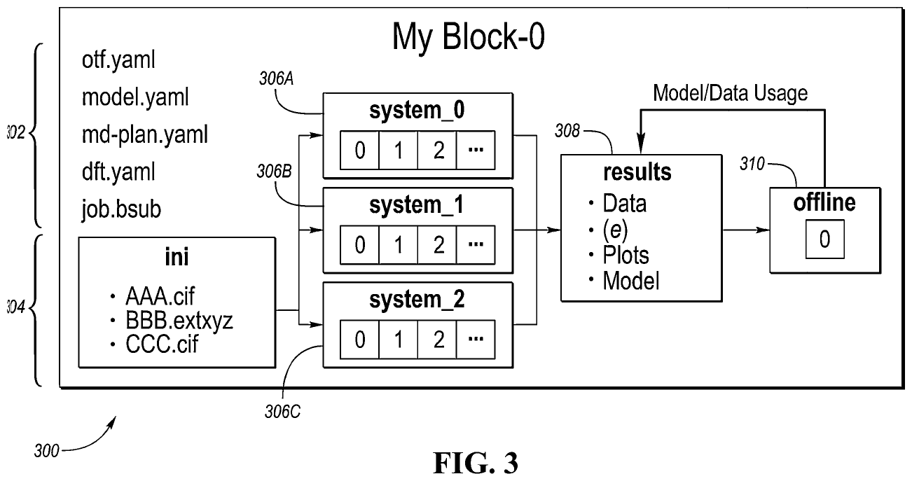

FIG. 3 is a schematic diagram of the functions of block 300 of an iterative GP-based MLIP training method according to one or more embodiments. Block 300 utilizes configuration files 302 (e.g., otf.yaml, model.yaml, md-plan.yaml, dft.yaml, and job.bsub). Ini files 304 (e.g., AAA.cif, BBB.extxyz, and CCC.cif) are input into system_0 306A, system_0 306B, and system_2 306C, which output results 308 (e.g., data, per atom energy estimated by that set of system <e>, plots, and models). Results 308 may be input into offline training 310. The output of offline training 310 may be input into results 308 for model and/or data usage.

The iterative GP-based MLIP training method of one or more embodiments may implement parallelization of training for multiple substructures on high-performance computing (HPC) facilities at both a project level and a block level. Multiple projects may be managed with submission of OTF training jobs for each system parallelly on the HPC. For instance, a set of substructures of water and NaCl structures 402 of workflow 400 in block 404 may be (1) bulk liquid H₂O, (2) bulk crystal H₂O, (3) bulk liquid H₂O with solvated Na⁺ and Cl⁻, and (4) bulk crystal NaCl, where all the substructures may have similar box sizes. Structures 402 may be created by a user of the GP-based MLIP training method. The substructures may be assigned to a system-level directory and an active learning loop may be conducted in each system folder. Jobs may be submitted to a HPC facility for each system at once where each system may have a distinct strategic dynamic trajectory such as temperature ramping or enhanced sampling.

After OTF/offline training in a block, the trained offline model can be inherited by a new block that starts with a set of more complex substructures. For example, after several iterations of the active learning loop in block 404, the offline model may be trained with all the training data found by the OTF training loop in each system. Block 406 may be created starting with a set of substructures such as (1) a 2×2×2 supercell of the previous bulk liquid H₂O in block “A”, (2) a liquid H₂O-crystal NaCl interface, (3) a 2×2×2 supercell of liquid H₂O with solvated Na⁺ and Cl⁻. The trained offline model in block 404 may be inherited by block 406 as a starting point for the actively learning loop in block 406.

The iterative GNN-based MLIP training method of one or more embodiments may include a set of scripts that use a GNN-based MLIP (e.g., NequIP or Allegro) to train a machine learning potential and identify hyperparameter, including determining and fixing instabilities in a given potential.

The iterative GNN-based MLIP training method may manage digital files and directories using a hierarchical order. The hierarchical order may be (1) a project level, (2) a block level, (3) a session level, and (4) an iteration level. The following discloses the functionality of each level according to one or more embodiments.

On the project level, the digital files and the digital sub-directories may be related to an application associated with a complex system that spans a broad spectrum of chemical elements and phases of materials. In one or more embodiments, a project may include a number of block-level directories.

On the block level, the digital files and sub-directories may correspond to one or more GNN-based MLIP training models (e.g., multiple NequIP and Allegro models). A block containing multiple models may be a block for hyperparameter screening. A block containing a single model may be a block for longer training and MD runs. Each of the blocks may contain multiple session-level directories (e.g., block 116 receiving input from dataset 118 from block 110). Workflow 100 also includes block 120, which receives data output from block 116.

On the session level, the digital files and the digital sub-directories may correspond to a NequIP/Allegro model with a fixed set of hyperparameters. A single NequIP/Allegro model may be trained iteratively and may be terminated mid-way if the performance is poor compared to other sessions.

On the iteration level, the digital files and the digital sub-directories may be related to a single NequIP/Allegro execution, which is a training restarted from the previous NequIP/Allegro execution. An iteration-level directory may contain a single training history and a temporary model ready for restart.

The iterative GNN-based MLIP training method of one or more embodiments may provide parallelized training for multiple hyperparameter sets on one or more HPC facilities. The iterative GNN-based MLIP training method of one or more embodiments may provide a flexible screening scheduler accepting the submission of an array of jobs in parallel where each job does the training on a candidate model with a unique hyperparameter set. The hyperparameter screening (e.g., as performed in block 408 of workflow 400) may be achieved through an iterative training loop where some candidate models are discarded if their performance is not Pareto efficient as disclosed herein. The performance may be defined by a set of user-defined metrics. After several iterative trainings, a model may be selected for longer training where all the candidate models are Pareto efficient. A non-limiting list of metrics of interest may include energy accuracy, force accuracy, stress accuracy, inference speed of the model, and graphics processing unit (GPU) memory footprint of model.

The iterative GNN-based MLIP training method of one or more embodiments may provide self-consistent iterative training that automatically adjust model defects (e.g., as performed in blocks 410 and/or 412 of workflow 400). A trained model may be sent to an array of stability tests by the iterative GNN-based MLIP training method. The iterative GNN-based MLIP training method may provide a flexible stability test scheduler that screens through all or some MD parameters (e.g., temperature, timestep, etc.). This test may result in nonphysical behavior, thereby creating atomic configurations that are not sufficiently represented in the training set. A subset of the test trajectory may then be selected and sent to a high-accuracy calculation such as DFT. These snapshots may be included in a training dataset for the next training iteration, possibly as a starting substructure for a block of an iterative GP-based MLIP training method, or as a single-point snapshot for an iterative GNN-based MLIP training session. This procedure may define a self-consistent iterative training loop. By means of this loop, the model may self-identify its defects and correct them along the iteration. When the hyperparameters are kept the same, adding these datapoints to the training set may resolve instabilities in the potential of the machine learning system.

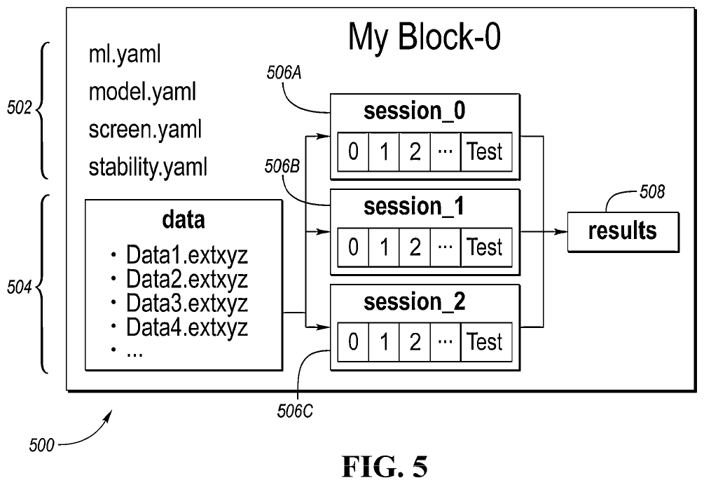

FIG. 5 is a schematic diagram of the functions of block 500 of an iterative GNN-based MLIP training method according to one or more embodiments. Block 500 utilizes configuration files 502 (e.g., ml.yaml, model.yaml, screen.yaml, and stability.yaml). Data files 504 (e.g., Data1.extxyz, Data2.extxyz, Data3.extxyz, and Data4.extxyz) are input into session_0 506A, session_1 506B, and session_2 506C, which produce results 508.

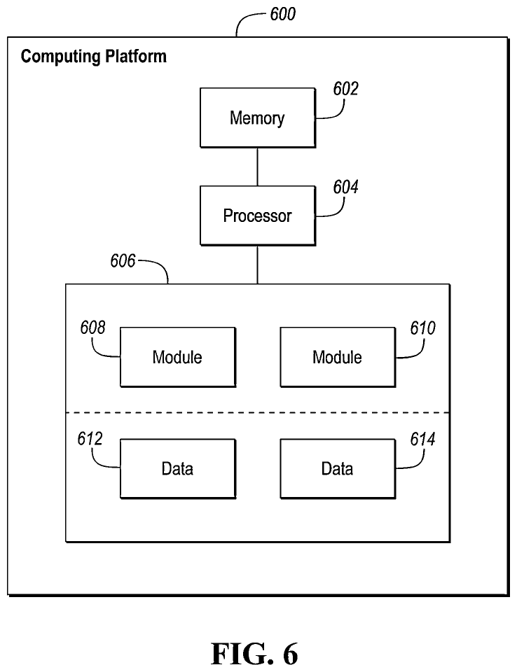

The iterative MLIP training methods of one or more embodiments are implemented using a computing platform, such as the computing platform 600 as illustrated in FIG. 6. The iterative MLIP training methods may be implemented as part of a computational software suite. The computing platform 600 may include memory 602, processor 604, and non-volatile storage 606. The processor 604 may include one or more devices selected from high-performance computing (HPC) facilities including high-performance cores, microprocessors, micro-controllers, digital signal processors, microcomputers, central processing units, field programmable gate arrays, programmable logic devices, state machines, logic circuits, analog circuits, digital circuits, or any other devices that manipulate signals (analog or digital) based on computer-executable instructions residing in memory 602. A HPC facility may include advanced computing hardware and software configured to perform computationally intensive tasks at much higher speeds than a typical desktop or server computer. The HPC facility may deliver up to one (1) million random read input/output operations per second (IOPS).

Memory 602 may include a single memory device or a number of memory devices including, but not limited to, random access memory (RAM), volatile memory, non-volatile memory, static random access memory (SRAM), dynamic random access memory (DRAM), flash memory, cache memory, or any other device capable of storing information. The non-volatile storage 606 may include one or more persistent data storage devices such as a hard drive, optical drive, tape drive, non-volatile solid state device, cloud storage or any other device capable of persistently storing information.

Processor 604 may be configured to read into memory 602 and execute computer-executable instructions residing in software modules 608 and 610 of the non-volatile storage 606 and embodying iterative MLIP training methods of one or more embodiments. Software modules 608 and 610 may include operating systems and applications. Software modules 608 and 610 may be compiled or interpreted from computer programs created using a variety of programming languages and/or technologies, including, without limitation, and either alone or in combination, Java, C, C++, C#, Objective C, Fortran, Pascal, Java Script, Python, Perl, and PL/SQL.

Upon execution by the processor 604, the computer-executable instructions of software modules 608 and 610 may cause the computing platform 600 to implement one or more of the iterative MLIP training methods of one or more embodiments disclosed herein. The non-volatile storage 606 may also include data 612 and 614 supporting the functions, features, and processes of the one or more embodiments described herein.

The program code embodying the algorithms and/or methodologies described herein is capable of being individually or collectively distributed as a program product in a variety of different forms. The program code may be distributed using a computer readable storage medium having computer readable program instructions thereon for causing a processor to carry out aspects of one or more embodiments. Computer readable storage media, which is inherently non-transitory, may include volatile and non-volatile, and removable and non-removable tangible media implemented in any method or technology for storage of information, such as computer-readable instructions, data structures, program modules, or other data. Computer readable storage media may further include RAM, ROM, erasable programmable read-only memory (EPROM), electrically erasable programmable read-only memory (EEPROM), flash memory or other solid state memory technology, portable compact disc read-only memory (CD-ROM), or other optical storage, magnetic cassettes, magnetic tape, magnetic disk storage or other magnetic storage devices, or any other medium that can be used to store the desired information and which can be read by a computer. Computer readable program instructions may be downloaded to a computer, another type of programmable data processing apparatus, or another device from a computer readable storage medium or to an external computer or external storage device via a network.

Computer readable program instructions stored in a computer readable medium may be used to direct a computer, other types of programmable data processing apparatus, or other devices to function in a particular manner, such that the instructions stored in the computer readable medium produce an article of manufacture including instructions that implement the functions, acts, and/or operations specified in the flowcharts or diagrams. In certain alternative embodiments, the functions, acts, and/or operations specified in the flowcharts and diagrams may be re-ordered, processed serially, and/or processed concurrently consistent with one or more embodiments. Moreover, any of the flowcharts and/or diagrams may include more or fewer nodes or blocks than those illustrated consistent with one or more embodiments.

The following application is related to the present application: U.S. patent application Ser. No. ______ (RBPA0467PUS) filed on ______, which is incorporated by reference in its entirety herein.

While all of the invention has been illustrated by a description of various embodiments and while these embodiments have been described in considerable detail, it is not the intention of the applicant to restrict or in any way limit the scope of the appended claims to such detail. Additional advantages and modifications will readily appear to those skilled in the art. The invention in its broader aspects is therefore not limited to the specific details, representative apparatus and method, and illustrative examples shown and described. Accordingly, departures may be made from such details without departing from the spirit or scope of the general inventive concept.

What is claimed is:

1. An iterative machine learning interatomic potential (MLIP) training method comprising:
training a first multiplicity of first MLIP models in a first iteration of a training loop;
training a second multiplicity of second MLIP models in a second iteration of the training loop in parallel with the first training step; and
combining the first MLIP models and the second MLIP models to create an iteratively trained MLIP configured to predict one or more values of a material.

2. The iterative MLIP training method of claim 1 further comprising predicting the one or more values of the material with the iteratively trained MLIP.

3. The iterative MLIP training method of claim 1, wherein the one or more values include total energy, atomic forces, atomic stresses, atomic charges, and/or polarization.

4. The iterative MLIP training method of claim 1 further comprising calling a third MLIP model when a predicted confidence of the first and/or second MLIP models falls below a predicted confidence threshold.

5. The iterative MLIP training method of claim 1 further comprising calling a third MLIP model when a predicted uncertainty of the first and/or second MLIP models exceeds a predicted uncertainty threshold.

6. The iterative MLIP training method of claim 1 wherein the first MLIP models use a first set of hyperparameters and the second MLIP models use a second set of hyperparameters different than the first set of hyperparameters.

7. The iterative MLIP training method of claim 1, wherein the first MLIP models use a first starting atomic structure and the second MLIP models use a second starting atomic structure different than the first starting atomic structure.

8. The iterative MLIP training method of claim 1, wherein the first MLIP models use a first chemical composition and the second MLIP models use a second chemical composition different than the first chemical composition.

9. The iterative MLIP training method of claim 1 further comprising selecting the iterative MLIP model from the model of the first and second MLIP models having the lowest error of the errors of the first and second MLIP models.

10. The iterative MLIP training method of claim 1, wherein the combining step accounts for an atomic environment overlap.

11. The interactive MLIP training method of claim 1, wherein the combining step accounts for the atomic changes in energies.

12. The iterative MLIP training method of claim 1, wherein the first training step produces a first machine learning system and the second training step produces a second machine learning system, and further comprising determining one or more instabilities in response to the first and second machine learning systems.

13. The iterative MLIP training method of claim 12 further comprising learning from the one or more instabilities when training a third MLIP model.

14. The iterative MLIP training method of claim 1 further comprising terminating the training loop when one or more of the models of the first and second MLIP models is not at least near a Pareto front.

15. The iterative MLIP training method of claim 1, wherein the first training step produces a first machine learning system, and further comprising generating at least one starting structure for an active learning scheme in the second training step in response to the first machine learning system.

16. The iterative MLIP training method of claim 1 further comprising removing datapoints based on a thermodynamic relevance.

17. An iterative machine learning interatomic potential (MLIP) training method comprising:
receiving one or more structures associated with a material;
training first MLIP models in a first training block using a first multiplicity of systems and the one or more structures associated with the material;
training second MLIP models in a second training block using a second multiplicity of systems; and
outputting one or more datasets from the second training block, the one or more datasets configured to predict one or more values of the material.

18. The iterative MLIP training method of claim 18, wherein the first and second MLIP models are first and second Gaussian Process (GP) based MLIPs.

19. An iterative machine learning interatomic potential (MLIP) training method comprising:
receiving one or more datasets associated with a material;
training first MLIP models in a first training block using a multiplicity of second sessions and the one or more datasets associated with the material;
training second MLIP models in a second training block using a second session; and
outputting one or more structures from the second training block, the one or more structures configured to predict one or more values of the material.

20. The iterative MLIP training method of claim 20, wherein the first and second MLIP models are first and second deep learning based MLIP models.
